(19) United States
(12) Patent Application Publication
(10) Pub. No.: US 2009/0317494 A1
(43) Pub. Date: Dec. 24, 2009

(54) Herbal composition containing COX-2 and its manufacturing method

(76) Inventors: Xiuying Lin, Anshun City (CN); Linxiang Mou, Anshun City (CN)

Correspondence Address: DAVID AND RAYMOND PATENT FIRM, 108 N. YNEZ AVE., SUITE 128, MONTEREY PARK, CA, 91754, US

(21) Appl. No.: 12/215,016

(22) Filed: Jun. 23, 2008

(51) Int. Cl. A61K 36/53; A61P 29/00

(52) U.S. Cl. 424/725

(57) ABSTRACT

A herbal composition, containing natural COX-2, includes a powder of Labiatae plant, wherein the herbal composition is prepared by a method including the steps of dehydrating the Labiatae plant and grinding the dried Labiatae plant into a powdered form. The herbal composition is used for treatment of a disease including Nephritis, Lymphoma and colorectal cancer, Acute, chronic, and bile-reflux cholecystitis, and aphonia. The herbal composition is formed as an internal treatment having an administrative form selected from the group consisting of capsule, powder, solution, and syrup. The herbal composition is formed as an external treatment by mixing the powdered Labiatae plant with “Vaseline”.

### BACKGROUND OF THE PRESENT INVENTION

1. Field of Invention

The present invention relates to herbal composition, and more particularly to a herbal composition containing COX-2 and its manufacturing method, which has notable effects on curing acute and chronic nephritis, acute and chronic cholecystitis (including bile-reflux cholecystitis), pathological changes of lymphatic system like lymphatic tumors or submandibular gland tumors, gastric tumors, mastitis, and the recovery after polyp surgery of either human bodies or throat.

2. Description of Related Arts

Cyclooxygenase-2, COX-2, was discovered in 1988. COX-2 is a protein acts as an enzyme to speed up the production of certain chemical messengers and is reputed to be effective in inflammation and pain. Accordingly, when COX-2 activity is blocked, inflammation is reduced.

According to Chinese (herbal) medicine, chemical compositions are extracted from natural herbs. Therefore, if we successfully find out and extract that functional chemical from the herbs, the patients can effectively take dose of concentrated functional chemical as the herbal medicine instead of having the whole plant of the herbs. Like the Western medicine, it is more efficient and effective.

### SUMMARY OF THE PRESENT INVENTION

A main object of the present invention is to a herbal composition containing natural COX-2 and its manufacturing method, which has notable effects on curing acute and chronic nephritis, acute and chronic cholecystitis (including bile-reflux cholecystitis), pathological changes of lymphatic system like lymphatic tumors or submandibular gland tumors, gastric tumors, mastitis, and the recovery after polyp surgery of either human bodies or throat.

Another object of the present invention is to provide a herbal composition containing natural COX-2 and its manufacturing method, wherein the herbal composition is extracted from a new and distinct variety of herbal Labiatae plant, which is a grass-like plant given the varietal denomination of ‘Guizhou Qizhen Cao’.

Another object of the present invention is to provide a herbal composition containing natural COX-2 and its manufacturing method, which has an administrative form selected from the group consisting of capsule, powder, solution, and syrup.

Another object of the present invention is to provide a herbal composition containing natural COX-2 and its manufacturing method, which can be formed as an external treatment by mixing the powdered Labiatae plant with “Vaseline”.

Another object of the present invention is to provide a herbal composition containing natural COX-2 and its manufacturing method, wherein the manufacturing method is easy and simply by making the Labiatae plant to a powder form.

Accordingly, in order to accomplish the above objects, the present invention provides a herbal composition containing natural COX-2, which comprises a powder of Labiatae plant.

A method of manufacturing a herbal composition containing natural COX-2, comprising the steps of:

(a) providing a Labiatae plant;

(b) dehydrating the Labiatae plant; and

(c) grinding the dried Labiatae plant into a powdered form.

The herbal composition has an administrative form of one of capsule, powder, solution, and syrup.

These and other objectives, features, and advantages of the present invention will become apparent from the following detailed description, the accompanying drawings, and the appended claims.

### BRIEF DESCRIPTION OF THE DRAWINGS

FIGS. 1 and 2 is a photograph of the new invention, ‘Guizhou Qizhen grass’, showing the whole plant.

FIG. 3 is a photograph of the invention showing the whole plant with roots.

FIG. 4 is a photograph of the invention showing its upright extending stem and flowers.

FIG. 5 is a photograph of the invention showing the appearance of its flowers in purplish red color.

FIG. 6 is a flow diagram illustrating a method of manufacturing a herbal composition containing natural COX-2 by Guizhou Qizhen grass.

### DETAILED DESCRIPTION OF THE PREFERRED EMBODIMENT

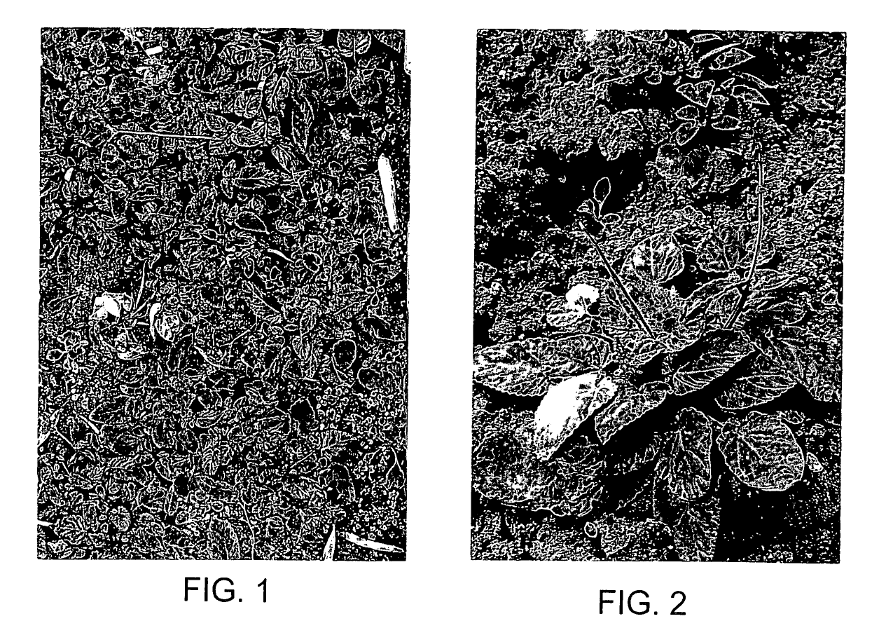

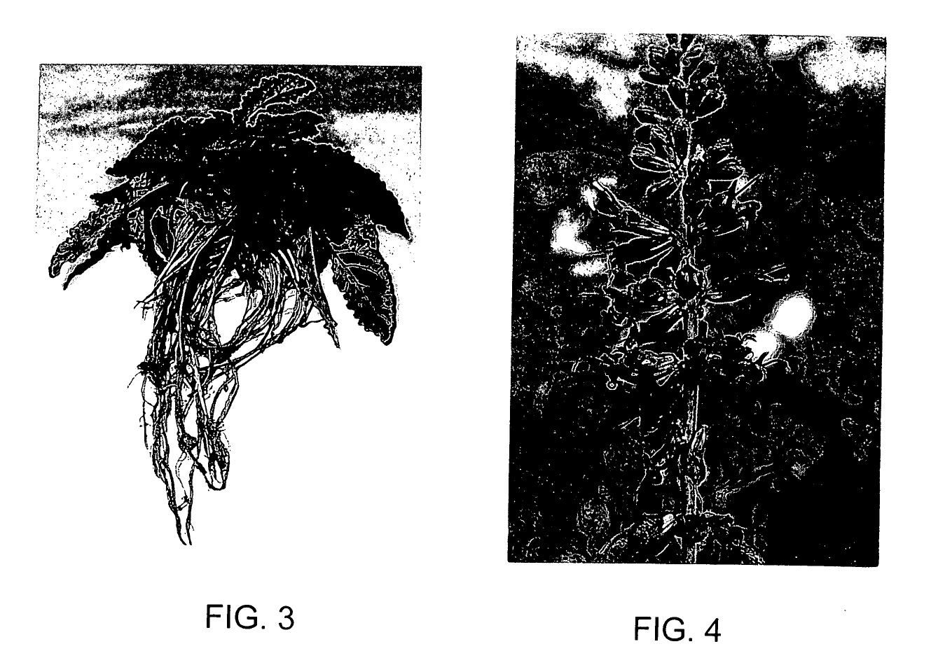

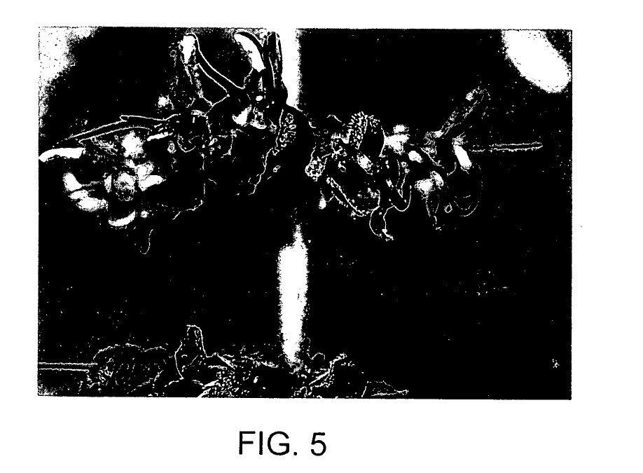

Referring to FIGS. 1 to 5, a herbal composition containing natural COX-2 and its manufacturing method is illustrated, wherein the herbal composition comprises a powder of Labiatae plant.

The new herb Guizhou Qizhen grass discovered by the applicant differs from related herbal plants including Sallvia Multiorrhiza, Bge (Dan Shen), Scutellaria Indica L. (Han Sin Cao), Scutellaria Barbata Don (Ban Zhi Lian), and Scutellaria Discolor Colebr (Wa Er Cao), in which the Dan Shen has pulp roots and white flowers, the Wa Er Cao has a soft hairy stem, fibrous roots and blue flowers, and the Ban Zhi Lian is arranged like a toothbrush that has fibrous roots and blue flowers.

The herbal Labiatae Plant, which is a grass-like plant, known as ‘Guizhou Qizhen Cao’. ‘Guizhou Qizhen Cao’ is a distinctive variety of Labiatae plant, which is characterized by its quadri-angular stem, fibrous roots with beaded root nodules grown thereon, purplish red peduncles, and purplish red flowers.

The descriptions disclosed herein are based upon observations of the plant growth.

The size of the whole plant has a height of 10-30 cm. The stem which has a quadric-angular shape is an erect stem or a stolon stem. The roots are fibrous roots with beaded root nodules grown thereon. The leaves are in oval shape and grown from the roots. Each leaf has a heart shaped leaf base and a peripheral edge with shallow round teeth. Each plant has single leaf or 1-2 pairs of compound leaf. Each leaf has a leafstalk having a length of 8-12 cm. The two blade surfaces of each leaf are hairless and have plenty of wrinkles. The color of the leaves is green or purplish red. The plant has about 30 leaves at the stem portion while the leaves at the peduncle are in pair manner. The flowers are arranged in circular taper manner and there are 15-22 rounds of flowers, wherein each round has 6 flowers and each flower has a size of 8×3 mm. The flowers are lip style flowers, each having a tubular shape and two slits at the lower lip. Each flower has two stamens. The style of each flower is grown from the ovary. The flowers are arranged at the top portion of the stem to form a whole series of flowers. The corolla of each flower is in purplish red color. The nuts are small and each has an elliptical shape and a dark red color. The nut is matured within the calyx.

The plant grows from seedling propagation and independently. The plant can be cultured and propagated in garden. The whole plant can be used as oriental medicine. The growing condition prefers windy, warm and wet environment. The rest period of the plant is winter that the plant stops to grow and to grow leaf temporally. The plant has a slight fragrant and a bitter taste. Each acre may grow more than 300-400 kg of fresh plants for two seasons per year. The plant is preferred to start planting in March of each year, in which it needs 40 days for budding and takes about 6 months from bud to mature for picking. The first time for seeding the plant is set between March and April and the second time for seeding the plant is set between September and October. Therefore, the plant gets mature between April and October. Preferably, the plant is seeded between May and June each year, wherein the time can then be set for next seeding after the harvest.

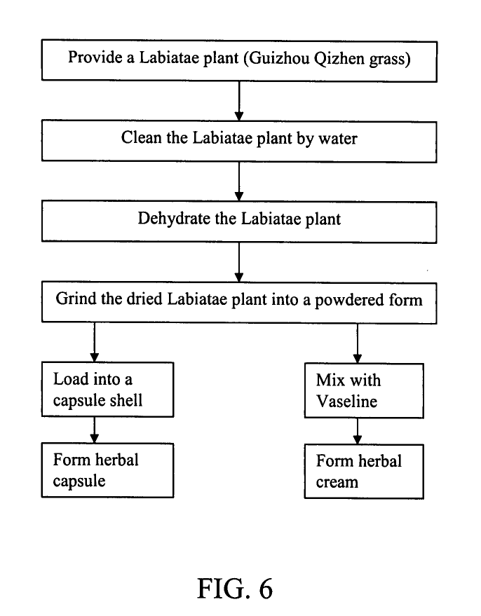

As shown in FIG. 6, the method of manufacturing the herbal composition containing natural COX-2 comprises the following steps.

(1) Provide the whole Labiatae plant (Guizhou Qizhen grass).

(2) Clean the Labiatae plant by water.

(3) Dehydrate the Labiatae plant.

(4) Grind the dried Labiatae plant into a powdered form, preferably by grinding machine.

According to the preferred embodiment, the herbal composition has an administrative form selected from the group consisting of capsule, powder, solution, and syrup.

In order for administration, the herbal composition further comprises a carrier loading with the powdered Labiatae plant, wherein the carrier comprises a capsule shell loading the powdered Labiatae plant therein to form a herbal capsule. The herbal capsule contains 0.35 g of the powdered Labiatae plant in the capsule shell.

The herbal composition is administrated in a dosage of 5.4 g per day for an adult for a treatment period ranging from 14 days to 42 days. In other words, the user will take the herbal composition three times a day and will take 1.8 g for each time. Therefore, the user will take five herbal capsules for each time. In particularly, the treatment period is set as seven days as one cycle. For acute inflammation, an adult will take two cycles, i.e. 14 days, as the treatment period. For chronic inflammation, an adult will take six cycles, i.e. 42 days, as the treatment period.

Alternatively, the carrier comprises “Vaseline” mixing with the powdered Labiatae plant to form a herbal cream as an external treatment to apply on an external injury area of a skin of a user, wherein a ratio of the powdered Labiatae plant and the “Vaseline” is 0.5:1 by weight.

Accordingly, the plant extract can rapidly subdue inflammation, swelling, and relieve pain. It can also eliminate viruses, inhibit toxic cells, activate the immune system, etc. After several years of observation on clinical application, the inventor discovered that Guizhou Qizhen grass has notable effects on curing acute and chronic nephritis, acute and chronic cholecystitis (including bile-reflux cholecystitis), pathological changes of lymphatic system like lymphatic tumors or submandibular gland tumors, gastric tumors, mastitis, and the recovery after polyp surgery of either human bodies or throat.

The ointment made of Guizhou Qizhen grass can cure sand holes and pinkeye; the external application can cure physical injury, burns from water or fire, sores and pus. If we stamp the original material and apply it to the body parts, it will help heal the fracture. If we add it to the Chinese herbal medicine and make it into facial ointment, it will beautify the skin through tightening, making the color lighter, removing wrinkles and brown or black freckles. By adopting Guizhou Qizhen grass, the above diseases will be cured rapidly and thoroughly without having a relapse, and the medicine even achieves 95% effectiveness. There are a lot more undiscovered applications; therefore, the inventor seeks to reserve the right for further study.

Below is brief introduction to some of the typical clinical cases:

I. Nephritis

1. Mr. Li., 65 years old, retired from Anshun in Guizhou. He had a nephrectomy that removed his right kidney. In October 2001, Mr. Li felt there was pain in the left portion of his waist, and then he found there was blood clot mixed in his urine. During that time, Mr. Li visited several local hospitals, including People's Liberation Army General Hospital. However, Mr. Li took the advice from mentioned hospitals' on bladder inspection rather than medical treatment. In May 2002, Mr. Li went to the inventor's place and tried the capsule made of Guizhou Qizhen grass. According to Mr. Li, there was not as much blood in his urine the day after he took the medicine, he did not feel as bloated in his waist anymore, and his urine went back to normal color on the third day. Mr. Lin had been taking the Guizhou Qizhen grass for two months, and he has not relapsed ever since then. He is in very good condition now.

2. Mr. Wei, 66 years old, retired from Anshun train station in Guizhou. He was in very bad health because he had suffered from chronic nephritis for several years. At that time, he only had about 0.1 lbs of rice every meal, and he felt fatigued and drowsy everyday; in addition, his feet had little strength, so he was immobile except during meals. After taking Guizhou Qizhen grass for half of a month in 2001, Mr. Wei's feet no longer felt bloated and weak, and he had better appetite. After one month of medication, Mr. Li's symptoms disappeared without any relapses.

3. Mr. Liu, 65 years old, retired from Anshun Xinhua bookstore in Guizhou. Mr. Liu started to feel bloated in his waist and found some blood in his urine in June 2007. The blood increased everyday. Being informed of the effectiveness of the medicine produced by the inventor, Mr. Liu turned to the inventor and tried Guizhou Qizhen grass. He told the inventor that the day after he took the medicine, “The blood in (my) urine has become two third less than before”, and he told the inventor again on the third day that the urine became clear and there was no more blood in it; meanwhile, the bloated feeling disappeared. Mr. Liu had taken the medicine for a month and had not relapsed.

4. Mrs. Liu, 60 years old, retired from an Anshun medicine company in Guizhou. In 2001, Mrs. Liu was admitted to a local hospital because of the following conditions: her facial and foot edema, abdominal distention, and difficulty urinating. Her symptoms were diagnosed as chronic nephritis with active break out because the urine test showed four “positive marks”. After consecutively injecting antibiotic medicine for more than 20 days; however, the urine test still showed four “positive marks”. Mrs. Liu started to take Guizhou Qizhen grass after she left the hospital, and meanwhile, she began to urinate more frequently. She had normal amount of urine after three days and she did not have the abdominal distention problem anymore. After she kept taking Guizhou Qizhen grass for 7 days, her facial and foot edema was cured. She took another urine test after 7 days, the original four “positive marks” all turned to be negative. Further, Mrs. Liu had no relapses after one month of taking the medicine. In addition, she can eat and sleep better than before she began the medication.

5. Dumei is a 24-year-old female from Zhejiang, Guizhou. In 2004, she started to feel bloated in her waist and found blood in her urine, and she felt fatigued and lost appetite. She visited local hospitals and experts in Chinese medicine from Shanghai, but had no improvement. She was referred to Anshun (where the inventor is located) by her relatives in 2005. Dumei had a urine test before the treatment and she had four “positive marks” shown in her occult-blood report, four “positive marks” in her corpuscle report, and three “positive marks” in her urine-protein report. She was very weak when she first visited the inventor. According to Dumei, her feet had little strength, she lost appetite, and she had medium foot edema-which is a typical syndrome of chronic nephritis. The day after she took Guizhou Qizhen grass, she said that there was less blood in her urine and she did not feel as bloated in her waist. Dumei kept taking the medicine. On the third day, her foot edema was less serious, she could urinate more while the color of her urine became lighter; she felt more energetic and she had more strength to walk. After taking the medicine for 12 days, Dumei went to have another urine test. This time, the report showed that she did not have blood in her urine, her white corpuscles and urine protein had become normal. Dumei continued taking the medicine for another two months and did not relapse. She is in good condition now.

II. Lymphoma and Colorectal Tumor

1. Mrs. Xiao, 64 years old, retired from an Anshun local governmental institute in Guizhou. In 1998, Mrs. Xiao found two lumps underneath her upper jaw confirmed by a doctor as submandibular gland tumors with a radius of 36.5×16 mm. Afterward, Mrs. Xiao visited several hospitals and tried all kinds of medicine, but without improvement. The lumps grew from 36.5×16 mm to 45.6 mm and were painful. Originally, Mrs. Xiao planned to have surgery, but decided to try treatment with Guizhou Qizhen grass instead. In less than 2 months, the lumps became smaller, from 45.6 mm to 13.9×9.1 mm and 9×10 mm, and disappeared after Mrs. Xiao completed one month of treatment. She has not relapsed and is in good health.

2. Mrs. Jiang, 40 years old, worked in Anshun train station in Guizhou. In 1996, Mrs. Jiang felt there was a lump in her neck, so she went to a hospital that confirmed she had a “chronic inflammation of neck organization” with “a lot of lymphatic tissue soaking.” Mrs. Jiang extremely worried about her disease, so she visited the city hospital and tried all kinds of medicine, but did not see improvement. Her lump grew as big as a chestnut and was painful, the soft tissue of her joints started to grow different sizes of lump; meanwhile, the tissue of her nasal cavity grew bigger and harder with irregular bleeding. Mrs. turned to the inventor and tried Guizhou Qizhen grass. Several days later, her nasal cavity stopped bleeding, the tissue of her nasal cavity became softer and the lumps disappeared after two months. Mrs. Jiang said that there was only a tiny lump in her neck by that time. She continued taking the medicine for a month, and she has been in good condition without any relapses.

3. Mr. Mei, 63 years old. In 1998, he started to have trouble defecating, and the blood jetted out in defecation. After the anal examination, he was confirmed to have a lump as big as a chestnut, located 15 cm from his anus. It did not improve after the medical treatment and the lump kept growing bigger. The excrement of the patient, as a result, became as thin as a pen with a lot of blood. Mr. Mei was very sick at that time. He started to take Guizhou Qizhen grass. On the third day, he stopped bleeding in defecation and it became a lot easier to defecate after half of a month; the size of the lump, according to Mr. Mei, became smaller. He continued to take the medicine for a month and he became healthier than before.

III. Acute, Chronic, and Bile-Reflux Cholecystitis

1. Mrs. Xu, 65 years old, retired from an Anshun knitwear mill in Guizhou. Mr. Xu had suffered from chronic cholecystitis for several years, and it broke out and was very painful as long as she did not take good care of her meals. Therefore, she could only have light meals and she always worried about the pain. In 1999, her son bought Guizhou Qizhen grass for her. She had taken the medicine for a month and had not relapsed ever since then. She does not worry about her meals anymore and is in good health.

2. Mrs. Yu, 62 years old, is from Pingba in Guizhou. She had suffered from bile-reflux cholecystitis for several years. She could not have any poultry, meat, and greasy food; the disease broke out often and was very painful. In 2003, her daughter brought her Guizhou Qizhen grass and Mrs. Yu had taken it for a month. She does not worry about the choice of food anymore and has not relapsed.

3. Mrs. Zhang, 50 years old, works in light industry in Guizhou. She had suffered from bile-reflux cholecystitis for several years, and that caused her to vomit a lot at parties. Mrs. Zhang used to have only half bowl of porridge and was very sick. In 2002, she took Guizhou Qizhen grass for two months, and now she is in good condition. Mrs. Zhang's daughter also had congenital cholecystitis, but recovered after taking the medicine for two months and has no relapsed.

4. Mrs. Chen, 52 years old, worked for Anshun medical company in Guizhou. She had suffered from lithiasis cholecystitis, coronary heart disease, hypertension, diabetes, and so on. Because the cholecystitis broke out frequently, Mrs. Chen had no choice but to be a vegetarian. She did not have enough nutrition and resistance to against the disease and she even had trouble going outside of the house. In 2003, she had taken Guizhou Qizhen grass and had not relapsed after half of a month. Mrs. Chen gradually recovered and she could go traveling after a month. She is currently in good condition.

IV. Aphonic After Throat Polyp Surgery

1. Mrs. Cai, 45 years old, is a manager of a construction company in Guizhou. In 2005, she had a laser treatment because of the polyp in her throat. After the treatment, she felt dry in her throat easily and had aphonia. Mrs. Cai tried all kinds of medical treatment, but no improvement. Her brother brought her Guizhou Qizhen grass from the inventor's place. On the day after she took the medicine, she no longer had problem speaking, but she had the same problem as soon as she stopped taking the medicine. She had taken the medicine for two months and her voice already recovered.

2. Ms. Wen, 26 years old, teaches in an Anshun kindergarten in Guizhou. In 1999, Ms. Wen had a laser treatment because of the polyp in her throat. She had aphonia for a long time after she left the hospital. After she took Guizhou Qizhen grass for a month, her throat did not feel uncomfortable anymore and her voice recovered. There were no relapses.

In 2006, Guizhou Qizhen grass was confirmed by the National Center for Drug Screening in China that it has natural COX-2 activity and immunoregulating activity which will cure inflammation. Meanwhile, the plant can control lymphatic cells T and B, so it has strong potential that need to be discovered.

The medicine is made of 1.8 grams per dose. A patient should take 5.4 grams of water-soluble medicine per day. The prescription for different disease works best when: the patient having acute nephritis and cholecystitis should take two treatment cycles (fourteen days), wherein each treatment cycle is seven days. The patient having chronic nephritis should take four treatment cycles, i.e. twenty-eight days. The patient having tumor should take eighteen treatment cycles, i.e. one hundred and fifty-six days. The patient having aphonia should take three treatment cycles, i.e. twenty-one days and there is no relapse. The medicine is prepared by the extracted Guizhou Qizhen grass and formed with petroleum jelly (“Vaseline”) for curing the eyes or external injury. The ratio of the medicine to petroleum jelly is 0.5:1 by weight.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A method of manufacturing a herbal composition containing natural COX-2, comprising the steps of:
(a) providing a Labiatae plant (Guizhou Qizhen grass);
(b) dehydrating said Labiatae plant; and
(c) grinding said dried Labiatae plant into a powdered form.

2. The method as recited in claim 1 wherein, before the step (b), said Labiatae plant is cleaned and washed by water.

3. The method, as recited in claim 2, further comprising a step of (d) loading said powdered Labiatae plant into a capsule shell to form a herbal capsule.

4. The method, as recited in claim 3, wherein said herbal capsule contains 0.35 g of said powdered Labiatae plant in said capsule shell.

5. The method, as recited in claim 2, further comprising a step of (d) mixing said powdered Labiatae plant with “Vaseline” to form a herbal cream.

6. The method, as recited in claim 5, wherein a ratio of said powdered Labiatae plant and said “Vaseline” is 0.5:1 by weight.

7. A herbal composition containing natural COX-2, comprising a powder of Labiatae plant (Guizhou Qizhen grass), wherein said herbal composition is prepared by a method comprising the steps of:
(a) providing said Labiatae plant;
(b) dehydrating said Labiatae plant; and
(c) grinding said dried Labiatae plant into a powdered form.

8. The herbal composition, as recited in claim 7, further comprising a carrier loading with said powdered Labiatae plant, wherein said carrier comprises a capsule shell loading said powdered Labiatae plant therein to form a herbal capsule.

9. The herbal composition, as recited in claim 8, wherein said herbal capsule contains 0.35 g of said powdered Labiatae plant in said capsule shell.

10. The herbal composition, as recited in claim 7, further comprising a carrier loading with said powdered Labiatae plant, wherein said carrier comprises “Vaseline” mixing with said powdered Labiatae plant to form a herbal cream.

11. The herbal composition, as recited in claim 10, wherein a ratio of said powdered Labiatae plant and said “Vaseline” is 0.5:1 by weight.

12. The herbal composition, as recited in claim 7, having an administrative form selected from the group consisting of capsule, powder, solution, and syrup.

13. The herbal composition, as recited in claim 7, which is administrated in a dosage of 5.4 g per day for an adult for a treatment period ranging from 14 days to 42 days.

14. The herbal composition, as recited in claim 7, which is administrated in a dosage of 5.4 g per day for an adult for acute inflammation with a treatment period of 14 days.

15. The herbal composition, as recited in claim 7, which is administrated in a dosage of 5.4 g per day for an adult for chronic inflammation with a treatment period of 42 days.

16. A method for the treatment of a disease treated with natural COX-2, comprising a step of administrating an effective amount of herbal composition containing Labiatae plant (Guizhou Qizhen grass), wherein said herbal composition is prepared by a method comprising the steps of:
(a) dehydrating said Labiatae plant; and
(b) grinding said dried Labiatae plant into a powdered form.

17. The method, as recited in claim 16, wherein said disease is selected from the group consisting of Nephritis, Lymphoma and colorectal tumor, Acute, chronic, and bile-reflux cholecystitis, and aphonia.

18. The method, as recited in claim 16, wherein said Labiatae plant is administrated in form of capsule by loading a powder form of said Labiatea plane into a capsule shell.

19. The method, as recited in claim 16, wherein said Labiatae plant is administrated in form of cream by mixing said powdered Labiatae plant with “Vaseline” to apply on an external injury area of a skin of a user.

20. The method, as recited in claim 16, wherein said Labiatae plant is administrated in form of capsule by mixing a powder form of said Labiatea plane with water.
